(19) United States
(12) Patent Application Publication
(10) Pub. No.: US 2012/0270778 A1
(43) Pub. Date: Oct. 25, 2012

(54) Compositions and Methods for Non-Invasive Treatment of Chronic Complications of Diabetes

(75) Inventor: Edward T. Maggio, San Diego, CA (US)

(73) Assignee: Aegis Therapeutics LLC

(21) Appl. No.: 13/514,720

(22) PCT Filed: Dec. 16, 2010
(86) PCT No.: PCT/US10/60900

§ 371 Date: Jul. 12, 2012

Related U.S. Application Data
(60) Provisional application No. 61/288,215, filed on Dec. 18, 2009.

(51) Int. Cl. A61K 38/00; A61P 3/10; A61K 38/28

(52) U.S. Cl. 514/6.5; 514/1.1

(57) ABSTRACT

The present invention provides C-peptide compositions that permit the noninvasive or non-injectable administration of C-peptide via nasal or pulmonary routes, as well as methods for treating disease.

### BACKGROUND OF THE INVENTION

1. Field of the Invention

The invention relates generally to pharmaceutical compositions and treatment of medical disorders and more specifically to treatment of diabetes with compositions including C-peptide.

2. Background Information

Type 1 diabetes (also called “insulin-dependent diabetes mellitus”, “brittle Diabetes” or “juvenile diabetes”) is the severe insulin-requiring form of diabetes usually affecting teens and under-30 adults, but which can affect infants or children. Type 1 diabetes is far less common than Type 2 diabetes, which typically affects older over-40 patients.

Common long-term complications of Type I diabetes include damage to the kidney called “nephropathy”, which if untreated can progress to chronic kidney failure (renal failure), requiring blood dialysis or kidney transplant. Retinal eye problems such as “diabetic retinopathy” or in the most serious form “proliferative diabetic retinopathy” (PDR) are also serious complication for diabetics. A neurological disorder common in Type 1 diabetes where the peripheral nerves are damaged is called peripheral nephropathy. The nerve damage can cause a number of symptoms including pain, loss of sensation and tingling. The feelings usually start in the peripheral areas such as the toes but may spread to the feet and hands.

Approximately 1 in 800 (or 0.12%) or 340,000 people are affected with Type 1 diabetes in the United States alone, with about 30,000 new cases diagnosed each year. More than one million people are currently affected by Type 1 diabetes worldwide. The first-line drug therapy for Type 1 diabetes is insulin administration. While insulin successfully controls blood glucose levels, it is less effective in controlling the chronic complications of diabetes over the long-term. As such, new drug formulations and methods of treatment of chronic complications of diabetes are sorely needed.

A growing number of therapeutic products are being applied to successfully control glucose levels in diabetic patients. The first line of therapy for treatment of type 1 diabetes is administration of insulin. Insulin is a peptide drug derived form a precursor protein produced in beta cells of the pancreas called proinsulin upon enzymatic cleavage into two pieces—insulin and C-peptide.

Since its discovery as a cleavage fragment of proinsulin, C-peptide has long been regarded as simply a means to insure proper folding of the insulin protein within the proinsulin precursor structure. More recently, it has been determined that C-peptide has important biological functions in preventing long-term complications of diabetes such as neuropathy and nephropathy, among others and increasing insulin sensitivity in Type I diabetic patients who have little or no naturally occurring C-peptide. Type 2 diabetics typically have circulating C-peptide in their blood although levels vary and some Type 2 diabetics have below normal C-peptide levels. Because insulin is a peptide drug, and because peptides are destroyed in the stomach when taken orally, insulin is administered by injection. While many Type 2 diabetic patients who produce some intrinsic insulin can delay the beginning of insulin therapy beyond the time at which many physicians feel that such therapy may be advisable to avoid having to stick themselves repeatedly with an insulin syringe multiple times per day, Type 1 diabetics must inject insulin to avoid drastic and lethal consequences.

The C-peptide fragment derived from pro-insulin upon liberation of insulin has been demonstrated to ameliorate many of the long-term chronic complications of diabetes. Unfortunately, like insulin, C-peptide is a bioactive peptide that must be administered in ways that avoid gastric hydrolysis and digestion in the stomach. Some peptides have been administered intranasally but with limited success. For example, salmon calcitonin, when administered as a metered nasal spray, results in only 3% systemic bioavailability. Similarly, desmopressin Nasal Spray when administered by the intranasal route yields bioavailability of between 3.3 and 4.1%.

Such low bioavailability requires undesirably high amounts of drug to be administered and results in high variability in serum drug levels. For example, while the average bioavailability of salmon calcitonin nasal spray is approximately 3%, the variability of concentration ranges from 0.3% to 30.6%—literally two orders of magnitude variability.

C-Peptide is a peptide which is produced when the pro-hormone pro-insulin is a enzymatically cleaved into insulin and C-peptide. While the role of insulin in controlling glucose levels has long been known, recently a growing number of studies in both animals and humans have demonstrated that C-peptide plays a role in preventing and potentially reversing some of the chronic complications of diabetes. For example, it has been shown that C-peptide improves neuropathy in a rat model of type 1 diabetes. It has also been reported that human clinical studies show that C-peptide administration in type 1 diabetes results in amelioration of diabetes-induced renal and nerve dysfunction. Further reports have demonstrated that C-peptide and the C-peptide fragment EVARQ reduce diabetes-induced hyperfiltration, as well as renal hypertrophy and albuminuria, a clinical indicator of diabetes introduced kidney damage.

In an exploratory, double-blinded, randomized, and placebo-controlled study, C-peptide treatment for 6 months improved sensory nerve function in patients with early-stage type 1 diabetic neuropathy. Others have demonstrated that C-peptide decreases islet cell apoptosis. C-peptide has also been shown to elicit disaggregation of insulin which increases the physiological effectiveness of insulin, and that C-peptide exerts antithrombotic effects that are repressed by insulin in normal and diabetic mice.

The results of these and other studies have prompted the hypothesis that C-peptide deficiency in diabetes may contribute to the development of various microvascular complications and that C-peptide replacement, together with regular insulin therapy, may be beneficial in treatment of prevention of these diabetic complications. U.S. Pat. No. 4,652,548 describes pharmaceutical formulations comprising human insulin, human C-peptide. These formulations are suitable for administration by injection or infusion and not intranasal administration. For example, they contain materials known to be toxic to nasal mucosal tissue such as phenol, meta-cresol and methyl-p-hydroxybenzoate. U.S. Patent Application Publication No. 20070082842 differentiates itself from U.S. Pat. No. 4,652,548 by claiming once-daily administration of C-peptide by subcutaneous injection. However, it is advantageous to if C-peptide administration and combinations of C-peptide and insulin are possible multiple times per day—preferably preprandially—to specifically mimic the natural-hormone secretion patterns that occur in response to food intake.

### SUMMARY OF THE INVENTION

The present disclosure is based upon the seminal discovery of aqueous compositions including C-peptide that are formulated for intranasal or pulmonary administration that provide high bioavailability of C-peptide as compared to other routes of administration.

Accordingly, in one embodiment the invention provides an aqueous composition of C-peptide or analog thereof which is formulated for intranasal or pulmonary administration. The composition includes C-peptide or an analog thereof; and a buffer solution. In various embodiments, the composition further includes insulin or an analog thereof. In some embodiments, the composition further includes one or more stabilizers, preservatives, penetration enhancers, and isotonicity adjustment agents. In some embodiments the pH is between about 4 and 8 and the buffer is acetate.

In another embodiment, the invention provides a method of administering C-peptide to a subject in need thereof. The method includes administering a composition as described herein to the subject via intranasal or pulmonary routes, thereby administering C-peptide to the subject.

In another embodiment, the invention provides a method of treating attenuated complications of diabetes of a subject. The method includes administering a composition as described herein to the subject via intranasal or pulmonary routes, thereby treating attenuated complications of diabetes of the subject.

In another embodiment, the invention provides a method of increasing insulin sensitivity in a subject. The method includes administering a composition as described herein to the subject via intranasal or pulmonary routes, thereby increasing insulin sensitivity in the subject.

### DETAILED DESCRIPTION OF THE INVENTION

The present invention is based on the discovery that C-peptides prepared in aqueous solution at moderate pH values ranging from about 4 to 8 or 4.5 to 7.5 are comparably well absorbed upon nasal administration yielding bioavailabilities in excess of 7 to 10% as compared to subcutaneous injection. As such, the present invention provides specific formulations that permit the noninvasive or non-injectable administration of C-peptide to diabetic patients and provides methods for ameliorating chronic complications of diabetes through noninvasive means.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

In one embodiment, the present invention provides aqueous formulations of C-peptide formulated for nasal administration. C-peptide is released systemically in stoichiometrically equivalent amounts to insulin since each pro insulin molecule is broken down into one insulin molecule and one C-peptide molecule. Since the transmucosal bioavailability of peptides administered intranasally is frequently less than the bioavailability obtained by intravenous or subcutaneous injection, or by pulmonary or nasal administration, the present invention provides for a range of concentrations of C-peptide in the aqueous formulations to allow for near stoichiometrically equivalent, amounts of insulin and C-peptide to be achieved in systemic circulation.

For example, the compositions described herein for pulmonary or intranasal delivery contain one or more of an aggregation inhibitory agent; a charge-modifying agent; a pH control agent; a degradative enzyme inhibitory agent; a mucolytic or mucus clearing agent; a ciliostatic agent; or a membrane penetration-enhancing agent.

Examples of membrane penetration-enhancing agents include cyclodextrins, such as methyl-beta-cyclodextrin; allcylglycosides, such as dodecylmaltoside and tetradecylmaltoside; an aggregation inhibitory agent; a charge-modifying agent; a pH control agent; a degradative enzyme inhibitory agent; a mucolytic or mucus clearing agent; a ciliostatic agent; a membrane penetration-enhancing agent selected from: (i) a cyclodextrin such as methyl-beta-cyclodextrin; an alkylglycoside or other surfactant; (ii) a bile salt; (ii) a phospholipid additive, mixed micelle, liposome, or carrier; (iii) an alcohol; (iv) an enamine; (v) an NO donor compound; (vi) a long-chain amphipathic molecule; (vii) a small hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); a modulatory agent of epithelial junction physiology; a vasodilator agent; a selective transport-enhancing agent; and a stabilizing delivery vehicle, carrier, mucoadhesive, support or complex-forming species with which the compound is effectively combined, associated, contained, encapsulated or bound resulting in stabilization of the compound for enhanced nasal mucosal delivery, wherein the formulation of the compound with the intranasal delivery-enhancing agents provides for increased bioavailability of the compound in a blood plasma of a subject.

Examples of preservatives that may be used in the compositions of the present invention, include, but are not limited to preservatives such as ethylene diamine tetraacetic acid (EDTA), sodium azide, p-hydroxybenzoate and its analogs, octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, chlorobutanol, m-cresol and alkyglycosides such as dodecyl maltoside.

In various embodiments, the compositions described herein may further include one or more excipients including stabilizers, surfactants, antimicrobial agents, osmolarity adjusting agents such as mannitol, sorbitol or sodium chloride.

The compositions described herein may include an acetate/acetic acid or citrate/citric acid buffer which may be buffered to have a pH of about 4 to 8, 4.5 to 7.5, 4.5 to 6.5, or 5 to 6.

As used herein, “alkylglycoside” refers to any sugar joined by a linkage to any hydrophobic alkyl, as is known in the art. Preferably the alkylglycoside is nonionic as well as nontoxic. Alkylglycosides are available from a number of commercial sources and may be natural or synthesized by known procedures, such as chemically or enzymatically.

In various aspects, alkylglycosides of the present invention may include, but not limited to: alkylglycosides, such as octyl-, nonyl-, decyl-, undecyl-, dodecyl-, tridecyl-, tetradecyl-, pentadecyl-, hexadecyl-, heptadecyl-, and octadecyl- α- or β-D-maltoside, -glucoside or -sucroside; alkyl thiomaltosides, such as heptyl, octyl, dodecyl-, tridecyl-, and tetradecyl-β-D-thiomaltoside; alkyl thioglucosides, such as heptyl- or octyl 1-thio α- or β-D-glucopyranoside; alkyl thiosucroses; alkyl maltotriosides; long chain aliphatic carbonic acid amides of sucrose β-amino-alkyl ethers; derivatives of palatinose and isomaltamine linked by amide linkage to an alkyl chain; derivatives of isomaltamine linked by urea to an alkyl chain; long chain aliphatic carbonic acid ureides of sucrose β-amino-alkyl ethers; and long chain aliphatic carbonic acid amides of sucrose β-amino-alkyl ethers.

As described above, the hydrophobic alkyl can thus be chosen of any desired size, depending on the hydrophobicity desired and the hydrophilicity of the saccharide moiety. For example, one preferred range of alkyl chains is from about 9 to about 24 carbon atoms. An even more preferred range is from about 9 to about 16 or about 14 carbon atoms. Similarly, some preferred glycosides include maltose, sucrose, and glucose linked by glycosidic linkage to an alkyl chain of 9, 10, 12, 13, 14, 16, 18, 20, 22, or 24 carbon atoms, e.g., nonyl-, decyl-, dodecyl- and tetradecyl sucroside, glucoside, and maltoside, etc. These compositions are nontoxic, since they are degraded to an alcohol or fatty acid and an oligosaccharide, and amphipathic. Additionally, the linkage between the hydrophobic alkyl group and the hydrophilic saccharide can include, among other possibilities, a glycosidic, thioglycosidic, amide, ureide, or ester linkage.

In sugar chemistry, an anomer is either of a pair of cyclic stereoisomers (designated α or β) of a sugar or glycoside, differing only in configuration at the hemiacetal (or hemiketal) carbon, also called the anomeric carbon or reducing carbon. If the structure is analogous to one with the hydroxyl group on the anomeric carbon in the axial position of glucose, then the sugar is an alpha anomer. If, however, that hydroxyl is equatorial, the sugar is a beta anomer. For example, α-D-glucopyranose and β-D-glucopyranose, the two cyclic forms of glucose, are anomers. Likewise, alkylglycosides occur as anomers. For example, dodecyl β-D-maltoside and dodecyl α-D-maltoside are two cyclic forms of dodecyl maltoside. The two different anomers are two distinct chemical structures, and thus have different physical and chemical properties. In one aspect of the invention, the alkylglycoside of the present invention is a β anomer. In an exemplary aspect, the alkylglycoside is a β anomer of an alkylmaltoside, such as tetradecyl-β-D-maltoside (TDM).

Thus, in one aspect of the present invention, the alkylglycoside used is a substantially pure alkylglycoside. As used herein a “substantially pure” alkylglycoside refers to one anomeric form of the alkylglycoside (either the α or β anomeric forms) with less than about 2% of the other anomeric form, preferably less than about 1.5% of the other anomeric form, and more preferably less than about 1% of the other anomeric form. In one aspect, a substantially pure alkylgycoside contains greater than 98% of either the α or β anomer. In another aspect, a substantially pure alkylgycoside contains greater than 99% of either the α or β anomer. In another aspect, a substantially pure alkylgycoside contains greater than 99.5% of either the α or β anomer. In another aspect, a substantially pure alkylgycoside contains greater than 99.9% of either the α or β anomer.

The buffered pharmaceutical compositions of the present invention are buffered such that upon nasal or pulmonary administration of the composition the pH of the nasal or pulmonary mucosa is relatively unperturbed. In various aspects, the pH of the nasal or pulmonary mucosa after administration remains unchanged or maintained within 1 pH point or less of the pH before administration of the pharmaceutical composition. Accordingly, the pH of the nasal or pulmonary mucosa after administration is maintained within less than 1, 0.9, 0.8, 0.7, 0.6, 0.5, 0.4, 0.3, 0.2 or 0.1 pH points of the pH before administration of the pharmaceutical composition.

The compositions of the present invention may further include insulin or an analog thereof in combination with C-peptide, Many types of insulin and functional analogs of insulin are known in the art are may be used in the present invention. For example, the C-peptide compositions may also include a recombinant human insulin. Such recombinant insulins may include sequence modifications that alter the pharmacokinetics or pharmacodynamics of the hypoglycemic effect to make the insulin short acting or long acting. Examples include Humalog®, Humulin® (Trademarks of Eli Lilly) and Novalog®.

Likewise, the compositions of the present invention include C-peptide or an analog thereof. Accordingly, the present invention provides compositions, including C-peptide fragments such as EVARQ.

In various embodiments, the C-peptide and the insulin are present in a ratio which allows substantially stoichiometrically equivalent concentrations of the C-peptide to the insulin. For example, the composition be formulated such that the C-peptide and the insulin are present in a ratio from about 20:1 to 0.4:1. In one embodiment, the invention provides aqueous therapeutic compositions in which a volume of 50 to 150 μL contains a stoichiometrically equal amount of C-peptide within a factor of 10, to the amount of insulin being administered. In yet another embodiment, the composition contains C-peptide and recombinant human insulin or sequence modified insulin.

The present invention also provides a method of treating diabetes or treating attenuated complications of diabetes including administering to a subject in need thereof via the nasal, inhalation or pulmonary routes, a pharmacologically effective amount of C-peptide alone or in combination with insulin, thereby reducing treating attenuated complications of diabetes such as neuropathy, reducing diabetes-induced renal dysfunction, reducing diabetes-induced hyperfiltration, reducing renal disfunction such as renal hypertrophy and albuminuria, decreasing islet cell apoptosis, increasing the physiological effectiveness of insulin, or countering the antithrombotic effects of exogenously administered insulin.

In another aspect, the invention provides a method of increasing insulin sensitivity in a diabetic subject by administering an aqueous composition having a therapeutically effective amount of C-peptide or C-peptide in combination with insulin.

As described herein, the compositions of the invention are formulated for nasal or pulmonary administration to a subject. The terms “administration” or “administering” as used herein are defined to include the act of providing a pharmaceutical composition of the invention to a subject in need of treatment. While the compositions described herein may be suitable for administration via any well known route, an exemplary administration route is nasal, intranasal or pulmonary administration. As used herein, the terms “nasal”, “intranasal” and “pulmonary” administration are intended to include administration to mucosal tissue lining the nasal cavity and the epithelial linings of the airway (e.g., trachea, bronchus, bronchioles and the like). Accordingly, in an exemplary aspect, the pharmaceutical compositions of the present invention, are formulated into pharmaceutically acceptable forms suitable for nasal and pulmonary administration, such as sprays and inhalants.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Intranasal administration of peptide drugs from aqueous solutions may be achieved by using any one of a number of commercially available metered nasal spray pumps. Manufacturers of such pumps include Pfeiffer and Valois. Dispensing volumes achievable by using these pumps range from approximately 50 μL up to 150 μL. Volumes beyond 150 frequently result in the drug running out of the patient's nostrils and unless a mucoadhesive or thickening agent is included in the formulation, volumes in excess of 150 μL are typically not used.

The dose of peptide drug, for example C-peptide, can be modulated by changing either the volume of spray admitted into the nostril or by changing the concentration of the peptide drug in the aqueous solution. The total amount of a C-peptide alone or C-peptide combined with insulin to be administered in practicing a method of the invention can be administered to a subject as a single dose or application (e.g., a single nasal application) over a relatively short period of time, or can be administered using a fractionated treatment protocol, in which multiple doses or applications are administered over a prolonged period of time. One skilled in the art would know that the amount of the peptide used to treat a subject depends on many factors such as the ailment or disease being treated, the age and general health of the subject as well as the number of treatments to be administered. In view of these factors, the skilled artisan would adjust the particular dose as necessary. In general, the dosage and frequency of administration are determined, initially, using Phase I and Phase II clinical trials. A suitable daily dose of a therapeutic peptide is generally that amount of the peptide which is the lowest dose effective to produce a therapeutic effect. Such an effective dose will generally depend upon the factors described above.

If desired, the effective daily dose of the therapeutic peptide may be administered as two, three, four, five, six or more sub-doses administered separately at appropriate intervals throughout the day, optionally, in unit dosage forms (e.g., single nasal applications). There may be a period of no administration followed by another regimen of administration.

It will be understood, however, that the specific dose level and frequency of dosage for any particular patient may be varied and will depend upon a variety of factors including the activity of the peptide employed, the metabolic stability and length of action of that compound, the age, body weight, general health, sex, diet, mode and time of administration, rate of excretion, drug combination, the severity of the particular condition, and the host undergoing therapy.

Accordingly, depending on the desired dosage for each application, the concentration of drug may be varied within the composition to allow for an appropriate amount of buffered solution to be delivered such that the pH of the nasal mucosa is unperturbed. Dispensing volumes lower than 50 μl by nasal pump is reported as being unacceptable as dose accuracy and probability of effective delivery to target organs is not assured; on the other hand, a higher volume, over 150 is considered in the art as unsuitable as this is known to lead to flooding. Accordingly, the compositions of the present invention may be administered at a single dose volume of from about 50 to about 150 μl; from about 75 to about 125 μl; from about 80 to about 110 μl; from about 85 to about 100 μl; or about 90 μl. The concentration of the peptide may be adjusted depending on the dose volume and desired amount of peptide to be delivered.

The following examples are intended to illustrate but not limit the invention.

### Example 1

### Preparation of Aqueous C-Peptide Formulations

Aqueous formulations of C-peptide are prepared by dissolving between 1 mg and 10 mg of C-peptide per mL in pH 5.5 sodium acetate buffer containing 0.9% sodium chloride. C-Peptide, human; Proinsulin C-Peptide (55-89), human is obtained from Biopeptide Inc. San Diego, Calif. Similarly, the corresponding EVARQ formulations are prepared using peptide as described by Ohtomo et al. (Diabetologia, 41:287-291 (1988)).

### Example 2

### Pharmacokinetic Analysis of Intranasally Administered C-Peptide in Rats

Test System—Sprague Dawley rats, females; 325 grams are used throughout this study. Rats are anesthetized by isoflurane/O₂ mixture. To a first and second group of three rats each is administered vehicle containing C-peptide concentration of 1 and 10 mg/mL as described in Example 1 to which is added 0.1% benzallconium chloride as a preservative. To a third and fourth group of 3 rats each is administered vehicle containing C-peptide concentration of 1 and 10 mg/mL as described above to which is added 45 mg/mL methyl-beta-cyclodextrin (Sigma-Aldrich). To a fifth group of 3 rats is administered vehicle containing EVARQ C-peptide fragment at a concentration of 10 mg/mL as described above to which is added 45 mg/mL methyl-beta-cyclodextrin. To a sixth group of 3 rats (the control group) the equivalent amount of C-peptide administered in the nasal 10 mg/mL test articles is administered as a single 100 microliter bolus injection to each rat above the femoris muscle. Rats are anesthetized by isoflurane/O2 mixture.

The nasal formulations were instilled into the left nare as a 25 microliter aliquot via micropipette to rats placed in the supine position. Following instillation, each rat was held in this position for an additional 10 seconds. Blood samples were collected from the orbital sinus after Isoflurane/oxygen inhalation for determination of the serum concentrations of C-peptide. Samples were collected, allowed to clot, and then stored on an ice block until centrifuged. Collection intervals are as follows: Predose (t₀) and at 15, 30, 45, 60, 120, 240 minutes post dose. Each sample is typically 0.5-1 mL. The test animals were not fasted before blood collection. After completion of blood collection at study termination, the animals were euthanized by carbon dioxide inhalation.

Analysis Procedure—The serum was frozen at approximately −70° C. in pre-labeled, plastic tubes, and tightly capped, and stored at approximately −70° C. until ready for testing. C-peptide concentrations are determined by C-Peptide ELISA Kit, Human, BioAssay™, United States Biological, Swampscott, Mass. or C-Peptide EIA (Enzyme immunoassay) Kit, High Sensitivity, Penninsula Labs, (Div. of Bachem, San Carlos, Calif. The EVARQ C-peptide fragment crossreacts sufficiently in the C-peptide enzyme immunoassay to permit measurement. EVARC standards were prepared by dissolving known amounts of EVARQ in normal rat serum. Area Under the Curve (AUC) from t=0 min to t=240 min. was measured. The ratio of AUC for the rats receiving nasal C-peptide to the AUC obtained for the subcutaneous injection control rats, adjusted for the relative amounts of C-peptide presented in each dose form (i.e., each test article or the control), is the relative nasal bioavailability of C-peptide.

Results are shown in Table 1 below.

TABLE 1

| Group | Test Article | Relative Bioavailability |
|---|---|---|
| 1 | Nasal C-peptide 1 mg/mL, bnz | 7%-18% |
| 2 | Nasal C-peptide 10 mg/mL, bnz | 8%-17% |
| 3 | Nasal C-peptide 1 mg/mL, bnz, | 12%-29% |
| | mbc | |
| 4 | Nasal C-peptide 10 mg/mL, bnz, | 12%-31% |
| | mbc | |
| 5 | Nasal EVARQ 10 mg/mL, bnz, | Approx. 40% |
| | mbc | |
| 6 | S.C. Injection Control | 100% (control) |

Key: bnz = benzalkonium chloride, mbc = methyl beta cyclodextrin

### Example 3

### Combined C-Peptide and Human Insulin Formulations of Defined Composition

Aqueous formulations containing both C-peptide and insulin were prepared as in Example 1, except that 5 millimolar acetate buffer was replace with 10 millimolar citrate buffer, pH 5.5 containing 0.125% dodecyl maltoside aggregation stabilizer. Insulin concentrations of 4 mg/mL and 20 mg/mL were prepared, to which are added C-peptide to yield final concentrations of C-peptide of 1, 3, 10, and 30 mg/mL C-peptide. Stability of the mixtures was determined by light scatter measurements. Solutions were incubated at room temperature (approximately 20° C.) on a rotary platform shaker (LabLine thermoregulated shaker) at 20 rpm for seven days. Protein aggregation was determined by measurements of light scatter using a Shimadzu RF-1501 recording spectrofluorophotometer with both the excitation and emission wavelengths set at 500 nm. Both excitation and emission wavelengths are set to 500 nm, and samples were read in disposable cuvettes with a 1 cm path length. For each reading, the instrument is zeroed with 1 ml of the appropriate buffer, then a 50 microliter aliquot of each formulation sample was added, mixed by inverting multiple times, and the cuvette checked for air bubbles before three stable readings were recorded. The spectrofluorophotometer is set for high sensitivity and the maximum possible reading is 1003 units. Measurements were taken daily during the seven day period.

After light scatter readings are taken, each protein sample is re-sealed with parafilm and returned to the shaker at 50 rpm, room temperature. All solutions remained completely clear and no increase in light scatter was observed over the seven data period indicating that mixtures of insulin and C-peptide are stable over this timeframe.

### Example 4

Combined C-Peptide and Human Insulin Formulations of Defined Composition. Insulin that Yield Stoichiometrically Equivalent Blood Levels of C-Peptide and Human Insulin Upon Intranasal Administration

Solutions prepared as described in Example 3 were administered nasally to rats and tested as described in Example 2 except with the addition that serum human insulin determinations were made using the Human Insulin ELISA Kit™ from Diagnostic Systems Laboratories, Inc, Webster, Tex. The relative molar ratios of insulin to C-peptide in serum for each formulation was calculated by taking the ratio of the insulin concentration to the C-peptide concentration at a fixed time point. In Table 2 below, ratios of the concentrations of insulin and C-peptide for concentrations measured at two time points, namely 20 min. and 40 min. post nasal instillation. The ratios shown below suggest that the C-peptide absorbs slightly, but not significantly, more rapidly than insulin. It can also be seen that certain ratios of insulin to C-peptide in the formulations can be chosen to allow stoichiometrically equivalent blood levels to be achieved shortly after administration. This is considered desirable since natural secretion of insulin from the pancreas results in stoichiometrically equivalent amounts of insulin and C-peptide released into systemic circulation. It is understood that differences in nasal mucosal tissue properties among different mammals may require different ratios than those shown below for rats and that species-specific ratios may be required for different species.

TABLE 2

| | | Relative molar ratios in | Relative molar ratios in |
|---|---|---|---|
| | | serum (Insulin:C-peptide) | serum (Insulin:C-peptide) |
| | | at 20 min. post nasal | at 40 min. post nasal |
| Group | Test Article | instillation | instillation |
| 1 | Nasal insulin 4 mg/mL, | 1:1 | 1:1 |
| | Nasal C-peptide 1 mg/mL | | |
| 2 | Nasal insulin 20 mg/mL, | 4:1 | 5:1 |
| | Nasal C-peptide 1 mg/mL | | |
| 3 | Nasal insulin 4 mg/mL, | 1:12 | 1:10 |
| | Nasal C-peptide 10 mg/mL | | |
| 4 | Nasal insulin 20 mg/mL, | 1:3 | 1:2 |
| | Nasal C-peptide 10 mg/mL | | |

### Example 5

### Formulations

The same experiment as described in Example 4 was performed with the following formulation to yield similar results.

FORMULATION

4 mg/mL recombinant human insulin (Eli Lilly)
1 mg/mL C-peptide
(The insulin to C-peptide ratio in formulation is 4 mg/mL to 1 mg/mL)
10 mM sodium citrate/citric acid buffer system, pH 4.5
45 mg/mL methyl-beta-cyclodextrin
1 mg/mL EDTA
1 mg/mL didecanoyl phosphatidylcholine
25 mM lactose
100 mM sorbitol
0.5% chlorobutanol

The same experiment as described in Example 4 was performed with the following formulation to yield similar results.

FORMULATION

4 mg/mL recombinant human insulin (Eli Lilly)
1 mg/mL C-peptide
(The insulin to C-peptide ratio in formulation is 4 mg/mL to 1 mg/mL)
10 mM sodium acetate buffer system, pH 5.9
1.8 mg/mL dodecyl-beta-D-maltoside
1 mg/mL EDTA

Although the invention has been described with reference to the above example, it will be understood that modifications and variations are encompassed within the spirit and scope of the invention. Accordingly, the invention is limited only by the following claims.

What is claimed is:

1. An aqueous composition comprising:
a) C-peptide or an analog thereof; and
b) a buffer solution, wherein the composition is formulated for intranasal or pulmonary administration.

2. The composition of claim 1, wherein the buffer is acetate.

3. The composition of claim 1, wherein the pH is between about pH 4 and pH 8.

4. The composition of claim 1, further comprising one or more of a stabilizer, a preservative, a penetration enhancer, and an isotonicity adjustment agent.

5. The composition of claim 1, wherein the composition further comprises an isotonicity adjustment agent selected from the group consisting of mannitol, sorbitol and NaCl.

6. The composition of claim 1, wherein the composition further comprises a penetration enhancer selected from the group consisting of methyl-beta cyclodextrin, EDTA, and an alkylglycoside.

7. The composition of claim 6, wherein the alkylglycoside is dodecyl maltoside or tetradecyl maltoside.

8. The composition of claim 1, wherein the composition further comprises a penetration enhancer selected from the group consisting of an alkylglycoside, benzalkonium chloride and chloroethanol.

9. The composition of claim 8, wherein the alkylglycoside is dodecyl maltoside or tetradecyl maltoside.

10. The composition of claim 1, further comprising insulin or an analog thereof.

11. The composition of claim 10, wherein the insulin is human insulin.

12. The composition of claim 10, wherein the C-peptide and the insulin are present in a ratio which allows substantially stoichiometrically equivalent concentrations of the C-peptide to the insulin.

13. The composition of claim 10, wherein the C-peptide and the insulin are present in a ratio from about 20:1 to 0.4:1.

14. The composition of claim 10, wherein the C-peptide and the insulin are present in a ratio which allows substantially stoichiometrically equivalent concentrations of the C-peptide to the insulin when measured within about 20 min to 60 minutes from nasal administration.

15. A method of administering C-peptide to a subject in need thereof comprising administering the composition of claim 1 to the subject via intranasal or pulmonary routes, thereby administering C-peptide to the subject.

16. The method of claim 15, wherein the subject has diabetes.

17. The method of claim 15, wherein the C-peptide is administered in metered dosage.

18. The method of claim 15, wherein the C-peptide is administered more than once per day.

19. A method of treating attenuated complications of diabetes of a subject comprising administering the composition of claim 1 via intranasal or pulmonary routes to the subject, thereby treating attenuated complications of diabetes of the subject.

20. A method of increasing insulin sensitivity in a subject comprising administering the subject the composition of claim 1 via intranasal or pulmonary routes, thereby increasing insulin sensitivity in the subject.
